(19) United States
(12) Patent Application Publication
(10) Pub. No.: US 2019/0156288 A1
(43) Pub. Date: May 23, 2019

(54) DOCUMENT MANAGEMENT SYSTEM

(71) Applicant: IRYOU JYOUHOU GIJYUTU KENKYUSHO CORPORATION, Fukouka (JP)

(72) Inventor: Shinkichi HIMENO, Fukuoka (JP)

(73) Assignee: IRYOU JYOUHOU GIJYUTU KENKYUSHO CORPORATION, Fukuoka (JP)

(21) Appl. No.: 16/098,967

(22) PCT Filed: Apr. 27, 2017
(86) PCT No.: PCT/JP2017/016652

§ 371 Date: Nov. 5, 2018

(30) Foreign Application Priority Data
May 12, 2016 (JP) 2016-095758

(51) Int. Cl. G06Q 10/10; G06F 16/93; G06F 16/28; G16H 40/20

(52) U.S. Cl. CPC G06Q 10/10; G16H 40/20; G06F 16/285; G06F 16/93

(57) ABSTRACT

[Problem] To promptly notify a change in an instruction document or record document pertaining to medical service, etc. to a staff who needs to know the change and eliminate the need for always monitoring changes, thereby making it possible to eliminate oversight and effectively utilize time accordingly. [Solution] A document management system provided with a case management means for managing a plurality of cases, a document category management means for managing a plurality of document categories, and a per-document category document recording means for recording a document for each document category. The document management system is provided with a trigger condition setting means for setting a trigger condition for triggering notification of the processing state of a document, a trigger condition applicable document detection means for detecting a document that falls under the trigger condition, a notified party setting means for setting a notified party to which the document detected to fall under the trigger condition is notified, and a trigger applicable document notification means for notifying the trigger applicable document detected to fall under the trigger condition by the trigger condition applicable document detection means to the notified party set by the notified party setting means.

### TECHNICAL FIELD

The present invention relates to a document management system that efficiently manages documents for instructions and records necessary when a large number of staff members work in parallel on a plurality of cases via a computer network, and especially to a document management system for an electronic medical record or the like which manages instructions and record documents relating to treatment or nursing of a patient at hospitals and the like.

### BACKGROUND ART

A large number of types of jobs such as medical doctors, nurses, caregivers, rehabilitators, pharmacists, and laboratory technicians are involved in the treatment and the like of patients. On the basis of a doctor's instruction document, a person of a type of job in charge confirms the instruction to prepare for and execute and creates a record document. Taking a blood examination involving multiple types of jobs as an example, a nurse draws blood and passes a sample to a laboratory technician and in turn the laboratory technician executes the examination and records a result thereof in a report document.

When there are multiple wards and examination instruction documents are sent from each ward, the laboratory technician creates his/her own work schedule (worksheet) and notifies each ward thereof. A nurse in a ward creates his/her own schedule based on schedules sent from each of other departments such as radiation department or physiological examination department so as to draw blood on the basis of this schedule and transfer a sample to the examination room.

In this manner, it is necessary for a large number of types of jobs to process diverse instruction documents in parallel in a distributed manner and keep results thereof in record documents. In working, it is essential to create a worksheet for each patient and for each type of job and proceed neatly with work. In addition, in a case where, for example, a work crosses multiple types of jobs, it is necessary to grasp the current progress status of the work such that the omission and duplication of implementation of the work are prevented.

Prior art literatures related to this application are as follows.

### CITATION LIST

### Patent Literature

Patent Literature 1: JP 11-066213 A

Patent Literature 2: JP 2012-033095 A

In Patent Literatures 1 and 2, related documents such as confirmation, implementation, and result reports are created for a medical instruction document such that it can be confirmed up to which stage the instruction has been implemented by which staff member. In addition, a worksheet is automatically created by consolidating the instruction documents for each ward and for each service department to make it possible to view the progress status of each instruction as a list by a technique such as changing the color of the document according to the creation stage of the related document.

### SUMMARY OF INVENTION

### Technical Problem

The patient's condition always changes. In accordance therewith, the instruction of medical care always changes.

That is, a new instruction document is suddenly created, or conversely, the content of the instruction document that has been issued is changed or deleted.

If these changes are missed, medical errors such as omission of implementation are brought about. Accordingly, medical staff members of each type of job need to constantly monitor whether no addition, change, or deletion has been made on instruction content, on a screen of the worksheet of electronic medical records. For this reason, a great deal of time has been wasted.

For blood examination or the like, it takes time to implement the examination and obtain the examination result since the instruction is issued.

In addition, for the instruction of the periodical examination, the result report document is created before a doctor who issued the instruction notices that. For this reason, many of the examinations that have been done carefully are not referred to in a timely manner and, in extreme cases, end up without being seen by the doctor at all. According to the result of a survey actually conducted at a US hospital, 40% of blood examinations, X-ray examinations, and the like end up without being referred to at the appropriate time.

Furthermore, it is estimated that this oversight will account also for 40% as a cause of medical accidents. Moreover, in order to avoid medical accidents due to such oversight, when an abnormal value is observed in a result of blood drawing or the like, the medical staff member is forced to confirm whether the doctor in charge has already referred to this result, which produces extra work.

The present invention has been made in order to solve such conventional difficulties and it is an object of the present invention to remove oversight by promptly notifying a staff member who needs to know a change in, for example, a medical instruction document or record document of this change to eliminate the need for constant monitoring of the change, making it possible to make effective use of the time saved therefrom, and furthermore, to provide a document management system in which a report on a result or the like of examination implemented carefully is surely referred to by a medical doctor and the like in a timely manner and the fact that the report has been referenced is promptly notified to a related medical staff member.

### Solution to Problem

As a means for achieving the above object, a document management system according to claim 1 is a document management system including: a case managing unit that manages a plurality of cases; a document category managing unit that manages a plurality of document categories; and a document-category-specific document recording unit that records a document for each document category, the document management system further including:

a trigger condition setting unit that sets a trigger condition serving as a trigger for notifying a processing state of a document;

a trigger condition corresponding document detecting unit that detects a document corresponding to the trigger condition;

a notification destination setting unit that sets a notification destination to be notified of a document detected as corresponding to the trigger condition; and

a trigger corresponding document notifying unit that notifies the notification destination set by the notification destination setting unit of a trigger corresponding document detected as corresponding to the trigger condition by the trigger condition corresponding document detecting unit.

A document management system according to claim 2 is the document management system according to claim 1, in which the trigger condition setting unit includes a document range setting unit that sets, as a range for setting the trigger condition, one of the following: (1) all of documents belonging to any of the document categories and related documents; (2) a particular document group among documents belonging to one of the document categories and a related document; and (3) a particular document among documents belonging to one of the document categories and a related document.

A document management system according to claim 3 is the document management system according to claim 1 or 2, in which the trigger condition setting unit includes a document processing state designating unit that allows to set at least one processing state among processing states of creation, update, and deletion of a document belonging to a range set by the document range setting unit, and creation, update, and deletion of a related document of the document.

A document management system according to claim 4 is the document management system according to any one of claims 1 to 3, in which the notification destination setting unit is enabled to set, as a notification destination, at least a particular staff member, a staff member belonging to a particular type of job, a staff member belonging to a particular work place, and any combination of the staff members.

A document management system according to claim 5 is the document management system according to any one of claims 1 to 4, in which the trigger corresponding document notifying unit is capable of using any one of a mail system or a sticky note system within the present document management system, an in-hospital mail system outside the present document management system, and a public mail system outside a hospital, or any combination of the foregoing systems.

A document management system according to claim 6 is the document management system according to any one of claims 1 to 5, in which the trigger corresponding document notifying unit includes a referring unit for the trigger corresponding document.

A document management system according to claim 7 is the document management system according to any one of claims 1 to 6, in which the trigger corresponding document notifying unit includes a notification read confirming unit that confirms that a relevant notification has been read at the notification destination.

### Advantageous Effects of Invention

Since the document management system according to claim 1 includes the trigger condition setting unit, when a document or a related document set by this trigger condition setting unit is created, changed, or deleted (document processing state designating unit), a trigger is activated and a notification is made.

Since the document management system according to claim 1 includes the trigger condition corresponding document detecting unit, this trigger condition corresponding document detecting unit detects change or deletion of a document or a related document set by the trigger condition setting unit.

Since the document management system according to claim 1 includes the trigger corresponding document notifying unit, this trigger corresponding document notifying unit notifies a notification destination set by the notification destination setting unit of the trigger corresponding document.

Since the document management system according to claim 2 includes the document range setting unit that sets a range of the trigger condition, the trigger condition can be set by designating any one range out of: (1) all of documents belonging to any of the document categories and related documents; (2) a particular document group among documents belonging to one of the document categories and a related document; and (3) a particular document among documents belonging to one of the document categories and a related document.

Since the document management system according to claim 3 includes the document processing state designating unit, at least one processing state can be set from among processing states of creation, update, and deletion of a document belonging to a range set by the document range setting unit, and creation, update, and deletion of a related document of the document.

In the document management system according to claim 4, since at least a particular staff member, a staff member belonging to a particular type of job, a staff member belonging to a particular work place, and any combination of the staff members can be set when a notification destination is set, a notification is made to an appropriate notification destination.

In the document management system according to claim 5, since the trigger corresponding document notifying unit is enabled to use any one of a mail system or a sticky note system within the present document management system, an in-hospital mail system outside the present document management system, and a public mail system outside a hospital, or any combination of the foregoing systems, an appropriate notification system is selectively applied.

Since the document management system according to claim 6 includes the referring unit for the trigger corresponding document, information related to a document notified by the trigger corresponding document notifying unit can be referred to.

Since the document management system according to claim 7 includes the notification read confirming unit, it can be confirmed that a notification notified by the trigger corresponding document notifying unit has been read at the notification destination.

### BRIEF DESCRIPTION OF DRAWINGS

FIG. 1 is an explanatory diagram of a patient ID master as a case managing unit.

FIG. 2 is an explanatory diagram of a document category managing unit.

FIG. 3 is an explanatory diagram of a document-category-specific recording unit.

FIG. 4 is an explanatory diagram of related document creation caused in the course of progression of an instruction document process and a notification destination that is triggered by the related document creation.

FIG. 5 is an explanatory diagram of a trigger condition and notification destination setting table.

FIG. 6 is an example of a notification document by a trigger corresponding document notifying unit.

### DESCRIPTION OF EMBODIMENTS

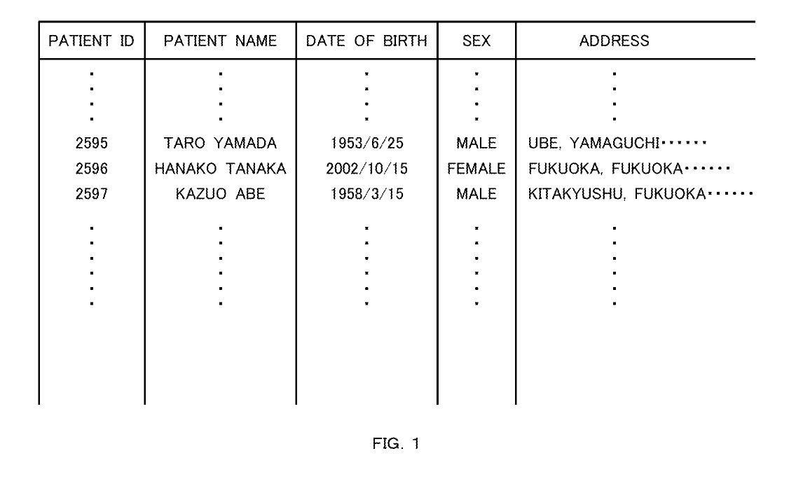

In FIG. 1, a patient ID master is taken here as an example of a case managing unit.

A unique management number (ID number) is assigned to each case or patient and all instruction documents and record documents are managed in association with this number. Personal information such as patient name and date of birth is also managed in this patient ID master.

Although medical records will be described below as an example, it goes without saying that the present invention is not restricted to electronic medical records but can also be applied to document management for project promotion, and the like.

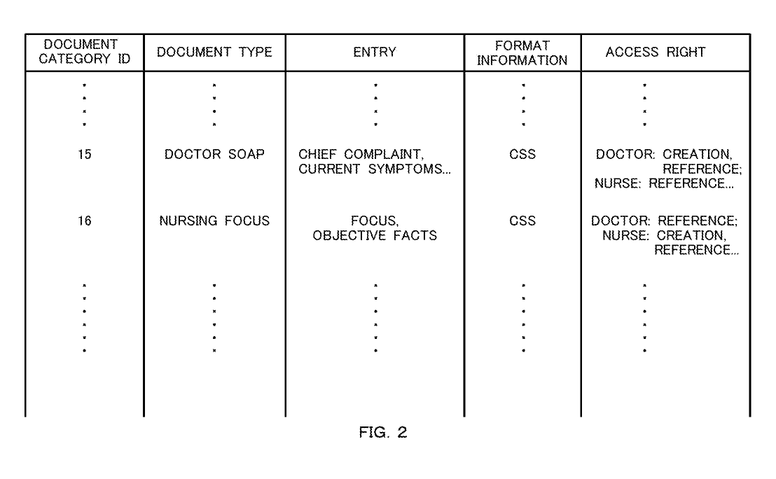

FIG. 2 is an explanatory diagram of a document category managing unit.

IDs of document categories created for each case (patient's medical record in this example), document type names, entries and format information included in documents, access rights of staff members, and the like are centrally managed for each document category.

Entries, formats, access rights, and the like are recorded as objects of an extensible markup language (XML) format in this example, but the present invention is not restricted thereto. A JavaScript object notation (JSON) format or the like may be employed as the expression format and the recording form is also optional, as long as the object of the present invention is met. Records may be composed for each entry to be recorded and managed using a relational database.

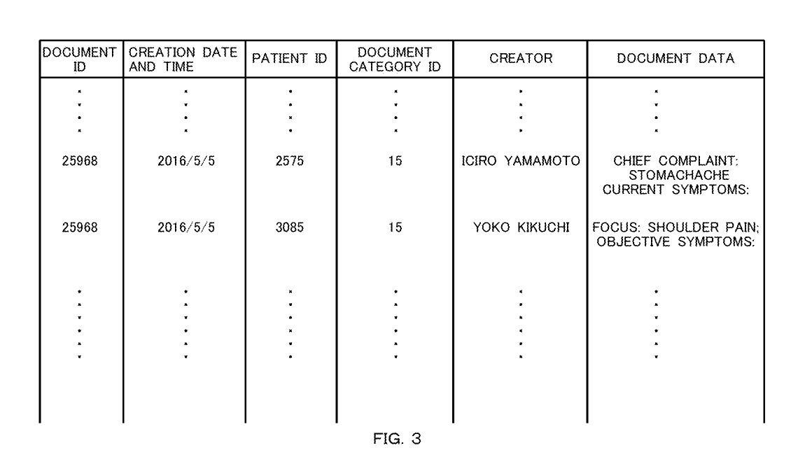

FIG. 3 is an explanatory diagram of a document-category-specific recording unit.

In this example, by allocating the ID of the document category, records are centrally managed for each document category entirely.

Search data such as a document ID as a serial number for management, a document creation date and time, a patient ID, a document category ID, a creator, and the like and object data in the XML format, which is the entity of document data, are recorded for each document.

As in the case of FIG. 2, in recording the document data in this case, the JSON format or the like may be employed as the expression format and the recording form is also optional. Records may be composed for each entry to be recorded and managed using a relational database.

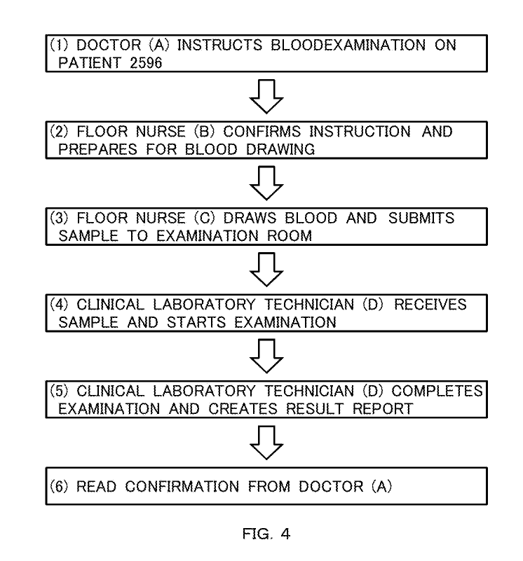

In FIG. 4, a flow of process and setting of a trigger condition and a notification destination will be described taking blood examination as an example.

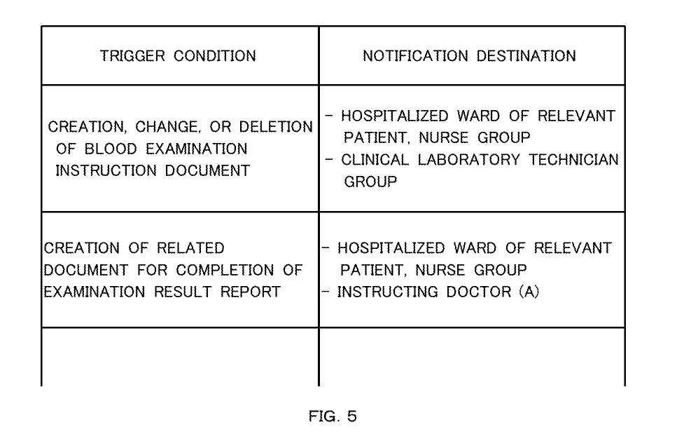

Here, FIG. 5 is a trigger condition and notification destination setting table created by a trigger condition setting unit and a notification destination setting unit.

When a document or a related document set by the trigger condition setting unit is created, changed, or deleted (document processing state designating unit), a trigger is activated and a notification is made.

(1) A doctor (A) creates a blood drawing instruction document for a patient ID 2596. The creation of this document is notified to a nurse group of a relevant ward where the patient is hospitalized, which is in charge of blood drawing, and a clinical laboratory technician group in charge of the examination (FIG. 5, the first row).

With this procedure, the floor nurses and the clinical laboratory technicians no longer need to constantly confirm whether an instruction document has been newly created or whether no change or deletion has been made, on a worksheet screen or the like, and can quickly begin taking actions such as preparation. At the same time, the risk of omission of implementation resulted from oversight is drastically reduced.

(2) Among the floor nurse group that was given the notification, a nurse (B) confirms the instruction and starts preparation for blood drawing, while creating a related document for instruction confirmation.

(3) A nurse (C) in charge of the relevant patient draws blood to deliver a sample obtained by blood drawing to a clinical examination room and at the same time creates a related document for blood drawing completion.

In this case, a triggered notification is not necessary because the delivery of the sample itself also serves as a notification.

(4) A clinical laboratory technician (D) who received the sample begins examining the sample.

(5) When the examination of the sample is finished, the clinical laboratory technician (D) inputs a result report document to an electronic medical record and also creates a related document for completion of the clinical examination.

The creation of this related document is notified to the doctor (A) who issued the instruction and the nurse group of the relevant ward according to the second row of FIG. 5. With this procedure, even if there is a time lag between the instruction creation and the result report, the result is referred to immediately without oversight and it is possible to contribute to the creation of an effective treatment policy.

(6) The doctor (A) who was given the notification opens the medical record of the relevant patient to refer to the examination result and also creates a related document for the fact that the examination result has been referred to.

This decreases the workload of floor nurses who had previously taken time to confirm whether the doctor had seen the results.

The trigger condition and notification destination setting table may be set only for a particular document. Usually, however, if the trigger condition and notification destination setting table is set by standard at the stage when the instruction document is created, for all of documents belonging to a certain document category and the related documents (for example, a blood examination instruction), or a particular document group and the related document (for example, documents relating to patients being hospitalized), or the like, this is useful as setting omissions are removed.

The notification destination is set by the notification destination setting unit. The notification destination can be set for a particular staff (the doctor (A) in this example), a staff member belonging to a particular type of job (nurse in this example), a staff member belonging to a particular work place (the ward where the patient is hospitalized in this example), and any combination of the above staff members.

Also in setting the notification destination as in the case of the trigger condition, if the notification destination is set by standard at the stage when the instruction document is created, for all of documents belonging to a certain document category and the related documents (for example, a blood examination instruction), or a particular document group and the related document (for example, documents relating to patients being hospitalized), or the like, this is useful as setting omissions are removed.

As described above, if the trigger condition and notification destination setting table is created every time an instruction document is newly created, a range for checking whether the trigger condition is satisfied when the state changes, such as when a certain related document is created, (trigger condition corresponding document detecting unit) is always maintained within a relevant document and the related document thereof, and the trigger condition and notification destination setting table set for the relevant document, such that the trigger condition can be checked in a minimum search range.

Nowadays when the performance of a computing device is improved, even a case where the trigger condition and notification destination setting table is separated to be managed and all documents are searched is also included in the scope of the present invention.

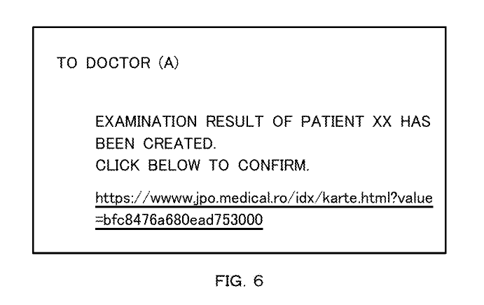

FIG. 6 is an example of the notification document transferred to a notification destination when it is determined that the trigger condition is satisfied. A script is embedded in the uniform resource locator (URL) and, when clicked, the electronic medical record of the relevant patient opens and the result of the blood examination can be referred to (referring unit for the trigger corresponding document).

In addition to simply displaying the electronic medical record of the relevant patient on a monitor screen in a usual manner, in this example, it is also useful to display the blood examination result or to create the related document for read confirmation in FIG. 4, by devising that script of the URL.

For the notification document, any of a message system or a sticky note system within an electronic medical record system, an e-mail system not included in the main body of the electronic medical record but running within a hospital, or an external e-mail system provided to the public may be used. However, the external e-mail system has security difficulties such as viruses and eavesdropping and it is thus preferable to use messaging/sticky note/e-mail system closed in the hospital.

Although an embodiment has been described so far, specific structures of the present invention are not limited to the above embodiment, and design changes and the like within the scope of the invention are included in the present invention.

For example, in the description of FIG. 4 and the following drawings, the present invention has been described based on the related document created according to the progression of the work and considered to be most useful. However, instead of creating the related document, a checklist or the like may be provided in the instruction document such that a check is put therein according to the working stage and this check put therein is used as a trigger. In addition, in FIG. 4, although it is assumed that the related document for read confirmation is created, a read confirmation function equipped in a general mail system may be used in the result report creation notification mail at the previous stage.

1. A document management system comprising: a case managing unit that manages a plurality of cases; a document category managing unit that manages a plurality of document categories; and a document-category-specific document recording unit that records a document for each document category, the document management system further comprising:
a trigger condition setting unit that sets a trigger condition serving as a trigger for notifying a processing state of a document;
a trigger condition corresponding document detecting unit that detects a document corresponding to the trigger condition;
a notification destination setting unit that sets a notification destination to be notified of a document detected as corresponding to the trigger condition; and
a trigger corresponding document notifying unit that notifies the notification destination set by the notification destination setting unit of a trigger corresponding document detected as corresponding to the trigger condition by the trigger condition corresponding document detecting unit.

2. The document management system according to claim 1, wherein the trigger condition setting unit includes a document range setting unit that sets, as a range for setting the trigger condition, one of the following: (1) all of documents belonging to any of the document categories and related documents; (2) a particular document group among documents belonging to one of the document categories and a related document; and (3) a particular document among documents belonging to one of the document categories and a related document.

3. The document management system according to claim 1, wherein the trigger condition setting unit includes a document processing state designating unit that allows to set at least one processing state among processing states of creation, update, and deletion of a document belonging to a range set by the document range setting unit, and creation, update, and deletion of a related document of the document.

4. The document management system according to claim 1, wherein the notification destination setting unit is enabled to set, as a notification destination, at least a particular staff member, a staff member belonging to a particular type of job, a staff member belonging to a particular work place, and any combination of the staff members.

5. The document management system according to claim 1, wherein the trigger corresponding document notifying unit is capable of using any one of a mail system or a sticky note system within the present document management system, an in-hospital mail system outside the present document management system, and a public mail system outside a hospital, or any combination of the foregoing systems.

6. The document management system according to claim 1, wherein the trigger corresponding document notifying unit includes a referring unit for the trigger corresponding document.

7. The document management system according to claim 1, wherein the trigger corresponding document notifying unit includes a notification read confirming unit that confirms that a relevant notification has been read at the notification destination.
